SEQUENCE LISTING (17 sequences)

SEQ ID NO: 1 - DNA, 90 bp, Homo sapiens
  CDS (1)..(90)
ttcctgactccgaaaaaactgcagtgcgttgacctgcacgttatctctaacgacgtttgcgctcaggttcacccgcagaaagttactaaa

SEQ ID NO: 2 - protein, 30 aa, Homo sapiens
FLTPKKLQCVDLHVISNDVCAQVHPQKVTK

SEQ ID NO: 3 - DNA, 210 bp, Artificial Sequence
  created by connecting the two copies of DNA molecules indicated in SEQ ID NO 1 using restriction enzymes and DNA ligase
ttcctgactccgaaaaaactgcagtgcgttgacctgcacgttatctctaacgacgtttgcgctcaggttcacccgcagaaagttactaaattcatgctgtgtagatccgagcctgaagagttcctgactccgaaaaaactgcagtgcgttgacctgcacgttatctctaacgacgtttgcgctcaggttcacccgcagaaagttactaaa

SEQ ID NO: 4 - protein, 70 aa, Artificial Sequence
  Synthetic Construct
FLTPKKLQCVDLHVISNDVCAQVHPQKVTKFMLCRSEPEEFLTPKKLQCVDLHVISNDVCAQVHPQKVTK

SEQ ID NO: 5 - DNA, 1752 bp, Artificial Sequence
  created by cloning the nucleotide with the sequence indicated in SEQ ID NO 1 to the 3′ terminus of HSP65 genes
atggccaagacaattgcgtacgacgaagaggcccgtcgcggcctcgagcggggcttgaacgccctcgccgatgcggtaaaggtgacattgggccccaagggccgcaacgtcgtcctggaaaagaagtggggtgcccccacgatcaccaacgatggtgtgtccatcgccaaggagatcgagctggaggatccgtacgagaagatcggcgccgagctggtcaaagaggtagccaagaagaccgatgacgtcgccggtgacggcaccacgacggccaccgtgctggcccaggcgttggttcgcgagggcctgcgcaacgtcgcggccggcgccaacccgctcggtctcaaacgcggcatcgaaaaggccgtggagaaggtcaccgagaccctgctcaagggcgccaaggaggtcgagaccaaggagcagattgcggccaccgcagcgatttcggcgggtgaccagtccatcggtgacctgatcgccgaggcgatggacaaggtgggcaacgagggcgtcatcaccgtcgaggagtccaacacctttgggctgcagctcgagctcaccgagggtatgcggttcgacaagggctacatctcggggtacttcgtgaccgacccggagcgtcaggaggcggtcctggaggacccctacatcctgctggtcagctccaaggtgtccactgtcaaggatctgctgccgctgctcgagaaggtcatcggagccggtaagccgctgctgatcatcgccgaggacgtcgagggcgaggcgctgtccaccctggtcgtcaacaagatccgcggcaccttcaagtcggtggcggtcaaggctcccggcttcggcgaccgccgcaaggcgatgctgcaggatatggccattctcaccggtggtcaggtgatcagcgaagaggtcggcctgacgctggagaacgccgacctgtcgctgctaggcaaggcccgcaaggtcgtggtcaccaaggacgagaccaccatcgtcgagggcgccggtgacaccgacgccatcgccggacgagtggcccagatccgccaggagatcgagaacagcgactccgactacgaccgtgagaagctgcaggagcggctggccaagctggccggtggtgtcgcggtcatcaaggccggtgccgccaccgacgtcgaactcaaggagcgcaagcaccgcatcgaggatgcggttcgcaatgccaaggccgccgtcgaggagggcatcgtcgccggtgggggtgtgacgctgttgcaagcggccccgaccctggacgagctgaagctcgaaggcgacgaggcgaccggcgccaacatcgtgaaggtggcgctggaggccccgctgaagcagatcgccttcaactccgggctggagccgggcgtggtggccgagaaggtgcgcaacctgccggctggccacggactgaacgctcagaccggtgtctacgaggatctgctcgctgccggcgttgctgacccggtcaaggtgacccgttcggcgctgcagaatgcggcgtccatcgcggggctgttcctgaccaccgaggccgtcgttgccgacaagccggaaaaggagaaggcttccgttcccggtggcggcgacatgggtggcatggatttcttcctgactccgaaaaaactgcagtgcgttgacctgcacgttatctctaacgacgtttgcgctcaggttcacccgcagaaagttactaaaaagcttgcggccgcactcgagcaccaccaccaccaccactga

SEQ ID NO: 6 - protein, 583 aa, Artificial Sequence
  Synthetic Construct
MAKTIAYDEEARRGLERGLNALADAVKVTLGPKGRNVVLEKKWGAPTITNDGVSIAKEIELEDPYEKIGAELVKEVAKKTDDVAGDGTTTATVLAQALVREGLRNVAAGANPLGLKRGIEKAVEKVTETLLKGAKEVETKEQIAATAAISAGDQSIGDLIAEAMDKVGNEGVITVEESNTFGLQLELTEGMRFDKGYISGYFVTDPERQEAVLEDPYILLVSSKVSTVKDLLPLLEKVIGAGKPLLIIAEDVEGEALSTLVVNKIRGTFKSVAVKAPGFGDRRKAMLQDMAILTGGQVISEEVGLTLENADLSLLGKARKVVVTKDETTIVEGAGDTDAIAGRVAQIRQEIENSDSDYDREKLQERLAKLAGGVAVIKAGAATDVELKERKHRIEDAVRNAKAAVEEGIVAGGGVTLLQAAPTLDELKLEGDEATGANIVKVALEAPLKQIAFNSGLEPGVVAEKVRNLPAGHGLNAQTGVYEDLLAAGVADPVKVTRSALQNAASIAGLFLTTEAVVADKPEKEKASVPGGGDMGGMDFFLTPKKLQCVDLHVISNDVCAQVHPQKVTKKLAAALEHHHHHH

SEQ ID NO: 7 - DNA, 1914 bp, Artificial Sequence
  created by cloning the nucleotide with the sequence indicated in SEQ ID NO 1 to the 3′ terminus of the nucleotide with the sequence indicated in SEQ ID NO 5
atggccaagacaattgcgtacgacgaagaggcccgtcgcggcctcgagcggggcttgaacgccctcgccgatgcggtaaaggtgacattgggccccaagggccgcaacgtcgtcctggaaaagaagtggggtgcccccacgatcaccaacgatggtgtgtccatcgccaaggagatcgagctggaggatccgtacgagaagatcggcgccgagctggtcaaagaggtagccaagaagaccgatgacgtcgccggtgacggcaccacgacggccaccgtgctggcccaggcgttggttcgcgagggcctgcgcaacgtcgcggccggcgccaacccgctcggtctcaaacgcggcatcgaaaaggccgtggagaaggtcaccgagaccctgctcaagggcgccaaggaggtcgagaccaaggagcagattgcggccaccgcagcgatttcggcgggtgaccagtccatcggtgacctgatcgccgaggcgatggacaaggtgggcaacgagggcgtcatcaccgtcgaggagtccaacacctttgggctgcagctcgagctcaccgagggtatgcggttcgacaagggctacatctcggggtacttcgtgaccgacccggagcgtcaggaggcggtcctggaggacccctacatcctgctggtcagctccaaggtgtccactgtcaaggatctgctgccgctgctcgagaaggtcatcggagccggtaagccgctgctgatcatcgccgaggacgtcgagggcgaggcgctgtccaccctggtcgtcaacaagatccgcggcaccttcaagtcggtggcggtcaaggctcccggcttcggcgaccgccgcaaggcgatgctgcaggatatggccattctcaccggtggtcaggtgatcagcgaagaggtcggcctgacgctggagaacgccgacctgtcgctgctaggcaaggcccgcaaggtcgtggtcaccaaggacgagaccaccatcgtcgagggcgccggtgacaccgacgccatcgccggacgagtggcccagatccgccaggagatcgagaacagcgactccgactacgaccgtgagaagctgcaggagcggctggccaagctggccggtggtgtcgcggtcatcaaggccggtgccgccaccgacgtcgaactcaaggagcgcaagcaccgcatcgaggatgcggttcgcaatgccaaggccgccgtcgaggagggcatcgtcgccggtgggggtgtgacgctgttgcaagcggccccgaccctggacgagctgaagctcgaaggcgacgaggcgaccggcgccaacatcgtgaaggtggcgctggaggccccgctgaagcagatcgccttcaactccgggctggagccgggcgtggtggccgagaaggtgcgcaacctgccggctggccacggactgaacgctcagaccggtgtctacgaggatctgctcgctgccggcgttgctgacccggtcaaggtgacccgttcggcgctgcagaatgcggcgtccatcgcggggctgttcctgaccaccgaggccgtcgttgccgacaagccggaaaaggagaaggcttccgttcccggtggcggcgacatgggtggcatggatttccatatggctagcgaattcgagcctgaagacttcctgactccgaaaaaactgcagtgcgttgacctgcacgttatctctaacgacgtttgcgctcaggttcacccgcagaaagttactaaattcatgctgtgtagatccgagcctgaagagttcctgactccgaaaaaactgcagtgcgttgacctgcacgttatctctaacgacgtttgcgctcaggttcacccgcagaaagttactaaattcatgctgtgcaagcttgcggccgcactcgagcaccaccaccaccaccactga

SEQ ID NO: 8 - protein, 637 aa, Artificial Sequence
  Synthetic Construct
MAKTIAYDEEARRGLERGLNALADAVKVTLGPKGRNVVLEKKWGAPTITNDGVSIAKEIELEDPYEKIGAELVKEVAKKTDDVAGDGTTTATVLAQALVREGLRNVAAGANPLGLKRGIEKAVEKVTETLLKGAKEVETKEQIAATAAISAGDQSIGDLIAEAMDKVGNEGVITVEESNTFGLQLELTEGMRFDKGYISGYFVTDPERQEAVLEDPYILLVSSKVSTVKDLLPLLEKVIGAGKPLLIIAEDVEGEALSTLVVNKIRGTFKSVAVKAPGFGDRRKAMLQDMAILTGGQVISEEVGLTLENADLSLLGKARKVVVTKDETTIVEGAGDTDAIAGRVAQIRQEIENSDSDYDREKLQERLAKLAGGVAVIKAGAATDVELKERKHRIEDAVRNAKAAVEEGIVAGGGVTLLQAAPTLDELKLEGDEATGANIVKVALEAPLKQIAFNSGLEPGVVAEKVRNLPAGHGLNAQTGVYEDLLAAGVADPVKVTRSALQNAASIAGLFLTTEAVVADKPEKEKASVPGGGDMGGMDFHMASEFEPEDFLTPKKLQCVDLHVISNDVCAQVHPQKVTKFMLCRSEPEEFLTPKKLQCVDLHVISNDVCAQVHPQKVTKFMLCKLAAALEHHHHHH

SEQ ID NO: 9 - DNA, 20 bp, Artificial Sequence
  artificialy synthesized based on the sequence of BCG HSP65 genes in the gene bank
ccatggccaagacaattgcg

SEQ ID NO: 10 - DNA, 18 bp, Artificial Sequence
  artificially synthesized based on the sequence of BCG HSP65 genes in the gene bank
gaaatccatgccacccat

SEQ ID NO: 11 - DNA, 82 bp, Artificial Sequence
  artificially synthesized by constricting a EcoR I site to the 5′ terminus of the nucleotide with the sequence indicated in SEQ ID NO 1
gccgagaattcgagcctgaagagttcctgactccgaaaaaactgcagtgcgttgacctgcacgttatctctaacgacgtttg

SEQ ID NO: 12 - DNA, 73 bp, Artificial Sequence
  artificially synthesized by constricting a BgI II site to the 5′ terminus of the nucleotide with the sequence indicated in SEQ ID NO 1
gggcgagatctacacagcatgaatttagtaactttctgcgggtgaacctgagcgcaaacgtcgttagagataa

SEQ ID NO: 13 - DNA, 81 bp, Artificial Sequence
  artificially synthesized by constricting a BamH I site to the 5′ terminus of the nucleotide with the sequence indicated in SEQ ID NO 1
gcgtggatccgagcctgaagagttcctgactccgaaaaaactgcagtgcgttgacctgcacgttatctctaacgacgtttg

SEQ ID NO: 14 - DNA, 73 bp, Artificial Sequence
  artificially synthesized by constricting a Hind III site to the 5′ terminus of the nucleotide with the sequence indicated in SEQ ID NO 1
cgccgaagcttgcacagcatgaatttagtaactttctgcgggtgaacctgagcgcaaacgtcgttagagataa

SEQ ID NO: 15 - DNA, 136 bp, Artificial Sequence
  created by using PCR primed by the primers with the sequence indicated in SEQ ID NO 11 and SEQ ID NO 12
gccgagaattcgagcctgaagagttcctgactccgaaaaaactgcagtgcgttgacctgcacgttatctctaacgacgtttgcgctcaggttcacccgcagaaagttactaaattcatgctgtgtagatctcgccc

SEQ ID NO: 16 - DNA, 134 bp, Artificial Sequence
  created by using PCR primed by the primers with the sequence indicated in SEQ ID NO 13 and SEQ ID NO 14
gcgtggatccgagcctgaagagttcctgactccgaaaaaactgcagtgcgttgacctgcacgttatctctaacgacgtttgcgctcaggttcacccgcagaaagttactaaatcatgctgtgcaagcttcggcg

SEQ ID NO: 17 - DNA, 246 bp, Artificial Sequence
  created by linking the nucleotides with the sequence indicated in SEQ ID NO 15 and SEQ ID NO 16 respectively
gaattcgagcctgaagagttcctgactccgaaaaaactgcagtgcgttgacctgcacgttatctctaacgacgtttgcgctcaggttcacccgcagaaagttactaaattcatgctgtgtagatccgagcctgaagagttcctgactccgaaaaaactgcagtgcgttgacctgcacgttatctctaacgacgtttgcgctcaggttccaccgcagaaagttactaaattcatgctgtgcaagctt

(19) United States
(12) Patent Application Publication
(10) Pub. No.: US 2006/0121050 A1
(43) Pub. Date: Jun. 8, 2006

(54) Fusion protein comprising bacillus calmette guerin heat shock protein 65 and the epitope of human prostate specific antigen

(76) Inventors: Liying Wang, Changchun (CN); Dapeng Li, Changchun (CN); Yongli Yu, Changchun (CN)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 10/635,187

(22) Filed: Aug. 6, 2003

(51) Int. Cl. A61K 39/02; C07H 21/04; C12N 15/74; C12N 1/21; C07K 14/32

(52) U.S. Cl. 424/190.1; 530/350; 435/69.3; 435/252.31; 435/471; 536/23.7

(57) ABSTRACT

Certain embodiments of the invention provide a fusion protein comprising Bacillus Calmette Guérin heat shock protein 65 and one to five copies of the epitope of human prostate specific antigen. The fusion protein provided by certain embodiments is therapeutic and/or preventive to human prostate cancer. Certain embodiments also provide the nucleic acid molecule encoding the fusion protein.

### CROSS REFERENCE TO RELATED APPLICATIONS

The present application is related to commonly owned and assigned U.S. patent application Ser. No. ______, filed Aug. 6, 2003, entitled “Recombinant Fusion Proteins Comprising BCG Heat Shock Protein 65 and the Epitope of MUC 1”, which is hereby incorporated by reference herein.

### FIELD OF THE INVENTION

The present invention relates to a recombinant fusion protein which is therapeutic and/or preventive to human prostate cancer. More particularly, the present invention relates to a recombinant fusion protein comprising Bacillus Calmette Guérin heat shock protein 65 and the epitope of human prostate specific antigen (hereinafter, it is also referred to as HSP65-PSAe). The present invention also relates to a nucleic acid molecule encoding the recombinant fusion proteins, a plasmid containing the nucleic acid molecule, a vaccine formulation comprising the recombinant fusion protein of the present invention as the active ingredient and a pharmaceutical acceptable excipient, and the use of the recombinant fusion protein of the present invention for preparing a pharmaceutical preparation for preventing and/or treating human prostate specific antigen expressing carcinomas.

### BACKGROUND OF THE INVENTION

Traditional strategies for therapy of human prostate cancer include operation, radiotherapy, chemotherapy and hormone therapy. However, each of the above strategies has its own limitations.

Human prostate specific antigen (PSA) is a tissue specific antigen expressed exclusively in the epithelial cells of prostate gland and prostate cancer cells. Research on PSA vaccine which is preventive and therapeutic to human prostate cancer has been conducted, such as constructing nucleic acid vaccine by cloning PSA gene into a mammalian expressing plasmid and this vaccine can induce humoral and cellular response against PSA (Kim J. J., et al., Molecular and immunological analysis of genetic prostate specific antigen vaccine. Oncogene. 1998, Dec. 17; 17(24): 3125-35). PSA-reactive immune cells were generated in patients with prostate cancer by using a mixture containing recombinant PSA and lipid A (Meidenbauer N., et al. Generation of PSA-reactive effector cells after vaccination with a PSA-based vaccine in patients with prostate cancer. Prostate. 2000, May 1; 43(2): 88-100).

### DESCRIPTION OF THE INVENTION

Materials and methods for creating an immune response to PSA are described herein, including embodiments for therapeutic, prophylactic, diagnostic, and research-tool purposes. An aspect of the invention is a molecule that has an element of PSA but also has an element that elicits an immune response in a patient such that the patient's immune system reacts to PSA. A preferred molecule is a recombinant fusion protein that has a portion that is similar to at least a portion of PSA and also has a portion that comprises at least a portion of Bacillus Calmette Guérin heat shock protein 65. Such a fusion protein will be useful for treating cancer, for reasons discussed below.

Cytotoxic T lymphocytes (CTL) are the most effective tumor killing cells in immune system. To stimulate anti-tumor immunity, a recombinant protein should be able to generate tumor specific CTLs. Usually, upon immunization, exogenously applied foreign proteins are taken up by and processed in MHC class II pathway in antigen presenting cells and subsequently activate humoral immune response (Heikema A, et al., Generation of heat shock protein-based vaccines by intracellular loading of gp96 with antigenic peptides. Immunol Lett., 1997, Jun 1;57(1-3): 69-74), but can not effectively induce the development of tumor-specific CTL, therefore can not be tumor preventive and therapeutic. So, conferring exogenously applied PSA with the property of specific CTL generating is a tactical approach for the developing PSA based recombinant vaccine which induces specific CTLs to human prostate cancer cells.

Heat shock protein (HSP) is a chaperon protein family present in a variety of creatures. In immune responses, HSP, as a CTL generating molecular adjuvant, assists the protein-antigen-uptake by dendritic cells and leads to the processing and presenting of protein-antigen in a MHC class I pathway. In addition, HSP is capable of stimulating dendritic cells to express co-stimulatory molecules (including B7) and secrete cytokines and therefore providing second signals for CTL activation.

In recent years, HSP based preparations have been demonstrated to be tumor-therapeutic and/or preventive. Treatment of mice with preexisting cancers with heat shock protein preparations derived from autologous cancer resulted in retarded progression of the primary cancer, a reduced metastasis of tumor cells, and prolongation of life-span of tumor bearing individuals (Tamura Y, et al., Immunotherapy of tumors with autologous tumor-derived heat shock protein preparations. Science, 1997, Oct. 3;278(5335):117-20). The growth of tumor cells of primary and metastasis of prostate cancer was significantly inhibited in mice with HSP-peptide complex derived from prostate cancer cells (Yedavelli SP, et al., Prevention and therapeutic effect of tumor derived heat shock protein, gp96, in an experimental prostate cancer model. Int. J. Mol. Med., 1999, Sep.; 4(3):243-8).

Since tumor cells can be inhibited using HSP-peptide complex derived from prostate cancer cells, the combination of HSP65 with a peptide analogous to PSA will elicit an immune response, both in vitro and in vivo. An HSP-tumor antigen fusion protein that contained PSA peptide(s) would be preventive and therapeutic for carcinomas, including human carcinomas.

Thus one embodiment of the invention provides a recombinant fusion protein which is therapeutic and/or preventive to human prostate cancer.

Another embodiment provides a nucleic acid molecule encoding the recombinant fusion protein of the present invention.

Another embodiment provides a plasmid containing the nucleic acid molecule of the present invention.

Another embodiment provides a vaccine formulation comprising the recombinant fusion protein of the present invention as the active ingredient and a pharmaceutical acceptable excipient.

Another embodiment provides a use of the recombinant fusion protein of the present invention for preparing a pharmaceutical preparation for preventing or treating human prostate specific antigen expressing carcinomas.

Embodiments of the invention are thus provided having a recombinant fusion protein comprising Bacillus Calmette Guérin heat shock protein 65 and 1-5 copies of the epitope of human prostate specific antigen (HSP65-PSAe) which is therapeutic and/or preventive to human prostate cancer. The epitope of human prostate specific antigen means a peptide fragment of the human prostate specific antigen (PSA) that can generate PSA specific cytotoxic T lymphocytes (CTL) and can be recognized by T cell receptor (TCR) of the CTL. The recombinant fusion protein may include one, two, three, four or five copies of the epitope of human prostate specific antigen. Preferably, the recombinant fusion protein includes one or two copies of the epitope of human prostate specific antigen. In the recombinant fusion protein of the present invention, the Bacillus Calmette Guérin heat shock protein 65 may be located at the N-terminus or C-terminus, preferably at the N-terminus, of the fusion protein. The epitope of human prostate specific antigen may be located at the C-terminus or N-terminus, preferably at the C-terminus, of the fusion protein.

In a preferred embodiment, the epitope of human prostate specific antigen has the amino acid sequence of SEQ ID NO: 2. Alternatively, the epitope has at least a portion of the amino acid sequence shown in SEQ ID NO: 2, or at least a portion of a sequence having 80%, 85%, 90%, or 95% identity to SEQ ID NO. 2. In another preferred embodiment, the epitope of human prostate specific antigen has the amino acid sequence of SEQ ID NO: 4. Alternatively, the epitope has at least a portion of the amino acid sequence shown in SEQ ID NO: 4, or at least a portion of a sequence having 80%, 85%, 90%, or 95% identity to SEQ ID NO: 4. Portion lengths may be, for example, at least 3 amino acids, at least 6, at least 8, less than 100, less than 50, less than 20, less than 15 amino acids, or any combination thereof.

In a preferred embodiment, the recombinant fusion protein has the amino acid sequence of SEQ ID NO: 6. Alternatively, the recombinant fusion protein has at least a portion of the amino acid sequence shown in SEQ ID NO: 6, or at least a portion of a sequence having 80%, 85%, 90%, or 95% identity to SEQ ID NO: 6. In another preferred embodiment, the recombinant fusion protein has the amino acid sequence of SEQ ID NO: 8. Alternatively, the recombinant fusion protein has at least a portion of the amino acid sequence shown in SEQ ID NO: 8, or at least a portion of a sequence having 80%, 85%, 90%, or 95% identity to SEQ ID NO: 8.

An embodiment set forth herein further provides a nucleic acid molecule encoding the recombinant fusion protein of the present invention. In a preferred embodiment, the nucleic acid molecule has the nucleotide sequence of SEQ ID NO: 5 or SEQ ID NO: 7. Alternatively, the nucleic acid molecule has at least a portion of the nucleotide sequence shown in SEQ ID NO: 5 or 7, or at least a portion of a sequence having 80%, 85%, 90%, or 95% identity to SEQ ID NO. 5 or 7.

An embodiment set forth herein also provides a plasmid containing the nucleic acid molecule of the present invention.

An embodiment set forth herein provides a vaccine formulation comprising the recombinant fusion protein of the present invention and a pharmaceutical acceptable excipient.

An embodiment set forth herein provides the use of the recombinant fusion protein of the present invention for preparing a pharmaceutical preparation for preventing and/or treating human prostate specific antigen expressing carcinomas. Preferably, the pharmaceutical preparation is a therapeutic and prophylactic vaccine.

Bacillus Calmette Guérin (BCG) heat shock protein 65(HSP65) is a BCG derived protein. After this protein is fused to one copy or two copies of the epitope of human prostate specific antigen, it can lead the epitope of human prostate specific antigen to enter into antigen presenting cells(APCs) including dendritic cells. The HSP65 assists the epitope of human prostate specific antigen to be processed via the endogenous route of antigen processing in the cells and the cells co-express the processed epitopes with MHC class I molecules on the surface of APCs which stimulate the generation of human prostate antigen specific cytotoxic T lymphocytes(CTL). The CTL will kill human prostate cancer cells expressing human prostate specific antigen. Furthermore, Bacillus Calmette Guérin (BCG) heat shock protein 65 can also stimulate antigen presenting cells (APCs) including dendritic cells to express co-stimulatory molecules (B7 molecules) and secrete cytokines. These costimulatory molecules and cytokines will enforce the tumor killing activities of CTL.

In preferred embodiments, a recombinant fusion protein as described herein can be administered to a human subcutaneously with the dosage between 100-500μg. To generate more and effective PSA specific cytotoxic T lymphocytes (CTL), 2 or more boosting immunization can be performed with an interval of 2 weeks to 2 month.

Other uses for fusion proteins described herein include uses as research tools, research reagents, and as agents for the creation of antibodies that may be used therapeutically, diagnostically, or as research reagents and tools. For example, the fusion proteins may be packaged, preferably with biologically acceptable agents and/or excipients, for use with in vitro in cell or tissue culture protocols to study aspects of immune system mechanisms. Antibodies to the fusion proteins are useful for identifying the patterns of localization of the fusion proteins in vitro or in vivo, and in cellular or tissue samples.

The identity of a protein or nucleic acid sequence is frequently established based on a sequence alignment of the DNA, RNA, or amino acids. Multiple alignments of such sequences are important tools in studying biomolecules. The basic information they provide is identification of conserved sequence regions. This is very useful in designing experiments to test and modify the function of specific proteins, in predicting the function and structure of proteins, and in identifying new members of protein families. Sequences can be aligned across their entire length (global alignment) or only in certain regions (local alignment). This is true for pairwise and multiple alignments. Global alignments with respect to polynucleic acids or polypeptides usually require gaps (representing insertions/deletions) while local alignments can usually avoid gaps by aligning regions between gaps. In a sequence alignment, letters arranged over one another are called matched. If two matched letters are equal, the match is called an identity otherwise the match is called a substitution or mismatch. An insertion or deletion is one or more letters aligned against a gap (−) and is considered the same as a mismatch for percent identity purposes (Waterman, M. S. 1995).

In some cases a determination of the percent identity of a peptide to a sequence set forth herein may be required. In such cases, the percent identity is measured in terms of the number of residues of the peptide, or a portion of the peptide. Thus a peptide of 10 residues would be 90% identical to SEQ ID NO 2 if nine of the residues of the peptide were determined to be matched to SEQ ID NO 2. A peptide or polypeptide of, e.g., 90% identity, may also be a portion of a larger peptide; for example, a peptide of 100 residues that has a portion that is 10 residues in length that is matched to 9 residues of SEQ ID NO 6 would have 90% identity with SEQ ID NO 6.

The amino acid residues described herein employ either the single letter amino acid designator or the three-letter abbreviation. Abbreviations used herein are in keeping with the standard polypeptide nomenclature, J. Biol. Chem., (1969), 243, 3552-3559. All amino acid residue sequences are represented herein by formulae with left and right orientation in the conventional direction of amino-terminus to carboxy-terminus.

### BRIEF DESCRIPTION OF THE DRAWINGS

Aspects of certain embodiments of the invention will be described in more detail with reference to the attached drawings in which:

FIG. 1 shows the purified recombinant HSP65-PSAe analyzed by SDS-PAGE. Lane a, b: bacterium lysate; lane m: protein marker; lane c, d, e: purified recombinant HSP65-PSAe.

FIG. 2 shows the generation of PSA specific CTL in mice by HSP65-PSAe.

FIG. 3 shows the growth inhibition of PSAe gene transfected tumor cells in mice injected with HSP65-PSAe.

FIG. 4 shows the generation of PSAe specific human CTL by in vitro immunization of autologous dendritic cells loaded with recombinant HSP65-PSAe.

### EXAMPLES

### Example 1

### Construction of BCG Heat Shock Protein 65 HSP 65 and Human Prostate Specific Antigen(PSA) Encoding Gene

Bacillus Calmette Guérin (BCG) was provided by Chang Chun Institute of Biological Products, China.

BCG genome DNA was extracted according to the methods as described in Molecular Cloning (J. Sambrook, Isolation of high-molecular weight DNA from mammalian cells, 9.16-9.22, Cold Spring Harbour Laboratory Press, Molecular Cloning, 1989).

The BCG heat shock protein 65 (HSP-65) encoding gene was amplified from a BCG genomic DNA by PCR using two primers (the forward primer, 5′ CCATG GCCAAGACAATTGCG 3′(SEQ ID NO: 9), and the reverse primer, 5′ GAAATCCATGCCACCCAT 3′(SEQ ID NO:10) ). The PCR reaction was performed according to following procedure: 94 , 30 seconds; 55 , 1 minute; 72 , 2 minutes; 30 cycles; a final extension at 72 for 10 minutes.

The BCG HSP-65 encoding gene was cloned into a pET28 vector (Novagen, U.S.A) using NcoI and EcoRI sites.

HSP65-PSA encoding genes were synthesized by the following procedures: Firstly, a DNA fragment with EcoRI and Bgl sites encoding a PSA derived peptide was synthesized in a PCR reaction using forward primer (5 ′GCCGAGAATTCGAGCCTGAAGAGTTCCTGACTC CGAAAAAACTGCAGTGCGTTGACCTGCACGTTATCTCTAACGACGTTTG3′(SEQ ID NO:11), with a EcoRI site) and reverse primer (5′ GGGCGAGATC TACACAGCAT GAATITAGTA ACTTTCTGCGGGTGA ACC TGA GCG CAA ACG TCG TTAGAGATAA 3′ (SEQ ID NO:12), with a Bgl _{II} site). The synthesized DNA was named as EcoRl-PSA-Peptide-BglII DNA fragment with the sequence of

(SEQ ID NO:15)

5′GCCGAGAATTCGAGCCTGAAGAGTTCCTGACTCCGAAAAAACTGCAGT
GCGTTGACCTGCACGTTATCTCTAACGACGTTTGCGCTCAGGTTCACCCG
CAGAAAGTTACTAAA TTCATGCTGT GTAGATCTCGCCC 3′

(one copy of the epitope of human prostate specific antigen). Secondly, another DNA fragment with Bgl and Hind III sites encoding a PSA derived peptide was synthesized in a PCR reaction using forward primer (5 ′ GCGTGGATCCGAGCCTGAAGAGTTCCTGACTCCGAAAAAACTGCAGTGCG TTGACCTGCACGTTATCTCTAACGACGTTTG3′(SEQ ID NO:13), with a BamHI site) and reverse primer (5′CGCCGAAGCTTGCACAGCATGAATTTAGTAAC TTTCTGCGGGTGA ACCTGA GCG CAA ACG TCG TTAGAGATAA 3′(SEQ ID NO: 14), with a HindIII site). The synthesized DNA was named as Bam HI-PSA-Peptide-Hind III DNA fragment with the sequence of 5′GCGTGGATCCGAGCCTGAAGAGTTCCTGACTCCGAAAAAACTGCAGTGC GTTGACCTGCACGTTATCTCTAACGACGTTTGCGCTCAGGTTCACCCGCAG AA AGTTACTAAATCATGCTGTGCAAGCTTCGGCG3′ (SEQ ID NO:16) (one copy of the epitope of human prostate specific antigen). Thirdly, the EcoRI-PSA-Peptide-BglII DNA fragment was digested with Bgl_{II} restriction enzyme and the Bam HI-PSA-Peptide-Hind III DNA fragment was digested with BamH_{I} restriction enzyme. The two digested DNA fragments were ligated to create a PSA derived peptide encoding DNA with the following sequence with a EcoRI site at its 5′ end and a HindIII site at its 3′ end: 5′GAATTCGAGCCTGAAGAG TTCCTGACTCCGAAAAAACTGCAGTGCGTTGACCTGCACG TTATCTCTAA CGACGTTTGCGCTCAGGTTCACCCGCAGAAAGTTACTAAATTCATGCTGT GTAGATCCGAGCCTGAAGAGTTCCTGACTCCGAAAAAACTGCAGTGCGTT GACCTGCACGTTATCTCTAACGACGTTTGCGCTCAGGTrCCACCGCAGAA AGTTACTAAATTCATGCTGTGCAAGCTT 3′ (SEQ ID NO: 17) (two copies of the epitope of human prostate specific antigen). The PSA derived peptide encoding DNA was digested with EcoR_{I} and Hind_{III} and cloned into the downstream of BCG HSP-65 encoding gene in pET28 vector using EcoR_{I} and Hind_{III} sites. The pET28 vector carrying HSP65-PSA derived peptides fusion proteins encoding gene was named as pET28-HSP65-PSA.

### Example 2

### Expression of Bacillus Calmette Guérin Heat Shock Protein 65—the Epitope of Human Prostate Specific Antigen Fusion Protein (HSP65-PSAe)

The bacterial clone producing HSP65-PSAe was obtained by the following procedures: Firstly, competent bacteria were prepared. The BL21 DE3(Novagen, U.S.A) bacteria were streaked onto the surface of an agar plate by using an inoculating loop. The inoculated plate was incubated at 37 for 12-16 hours. A single bacterium colony was picked into 2 ml of LB medium in a 15 ml tube and the tube was agitated vigorously for 1 minute. 1 ml of the bacteria suspension was transferred into 100 ml LB in a 1-liter flask, followed by incubation at 37 with vigorous agitation (225 r/min) until the medium OD₆₀₀ reaches 0.5 (within about three hours). The cultured bacteria were cooled on ice for 2 hours and collected by centrifugation at 2,500 g at 4 for 20 minutes. The bacterial pellet was resuspended in 100 ml ice-cold Trituration buffer (100 mmol/L CaCl₂, 70 mmol/L MgCl₂, 40 mmol/L Acetate Acid, PH5.5). The bacteria were incubated on ice for 45 minutes, followed by centrifugation at 1,800 g at 4 for 10 minutes. The bacterial pellet was resuspended in 10 ml ice cold Trituration buffer and a 200 μl aliquot of the bacterium suspension with 15% glycerol was dispensed into a sterile Eppendorf tube. The bacteria in the Eppendorf tube were competent cells for transformation of plasmid and were stored at −70.

Secondly, pET28-HSP65-PSA plasmids carrying HSP65-PSAe encoding genes were transformed into BL21 DE3 bacteria. A 200μl aliquot of competent cells in an Eppendorf tube was thawed on ice. 3 μl DMSO and 0.5 g pET28-HSP65-PSA plasmids carrying HSP65-PSAe encoding genes were added and mixed gently. The bacteria in the tube were incubated on ice for 30 minutes and on a rack in a preheated 42 circulating water baths for 45 seconds. The bacteria in the tube were allowed to cool on ice for 1-2 minutes and then transferred into a flask containing 2 ml LB medium, followed by incubation at 37 with rotation at 225 r/min for 1 hour. The bacteria were collected by centrifugation at 4,000×g for 5 minutes and were resuspended in 200 μl LB medium. An appropriate volume of transformed competent cells was spread onto an agar plate containing Kanamycin (50 μg/ml). The plate was inverted and incubated at 37 for 12-16 hours.

Thirdly, the HSP65-PSAe producing bacterium clone was collected, identified by restriction enzyme digestion-agarose gel electrophoresis and DNA sequencing, freeze-dried and stored at −20.

A single bacterium colony already identified was picked into 10 ml LB medium in a 250 ml flask, and was incubated at 37 with 225 r/min vigorous agitation until the bacteria OD₆₀₀ reaches 0.7. The resultant bacteria in the 10 ml were inoculated into 10 liter LB medium supplemented with glucose (2 g/L). The bacteria were cultured at 37 with 225 r/min vigorous agitation. When the bacteria OD₆₀₀ reaches 1.0, IPTG was added with the final concentration of 0.4 mM to induce the expression of HSP65-PSAe for 4 hours. The resultant bacteria were harvested by centrifugation at 4 for 15 minutes.

### Example 3

### Purification of Bacillus Calmette Guérin Heat Shock Protein 65-the Epitope of Human Prostate Specific Antigen Fusion Protein (HSP65-PSAe)

The E. coli cells were thawed on ice, suspended in urea and then lysed by using a mechanical method. The Bacillus Calmette Guérin heat shock protein 65-the epitope of human prostate specific antigen fusion protein (HSP65-PSAe) in the lysate was purified through Nickel affinity chromatography, hydrophobic chromatography and ion-exchange chromatography successively.

After being sterilized through 0.2 μM membrane filtration, the purified recombinant HSP-PSAe fusion protein was stored at −70.

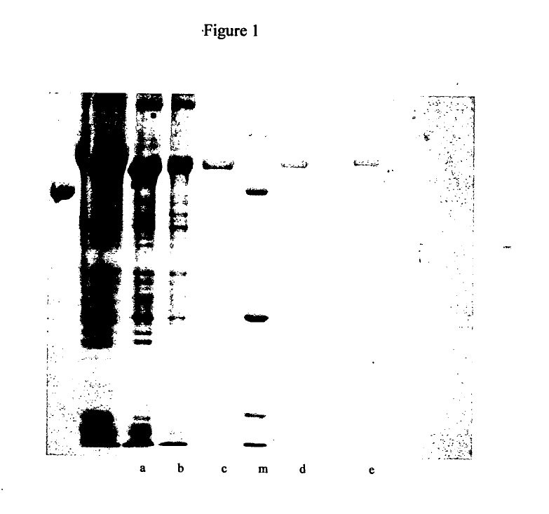

The purity of the fusion protein was analyzed by SDS-PAGE methods as described in Molecular Cloning (J. Sambrook, Polyacrylamide gel electrophoresis 6.36-6.49,Cold Spring Harbor Laboratory Press, Molecular cloning, 1989). SDS-PAGE analysis of the recombinant HSP65-PSAe is shown in FIG. 1. The result shows that the purity is over 96%.

### Example 4.

### Induction of PSA Specific Cytotoxic T Lymphocytes with Bacillus Calmette Guérin Heat Shock Protein 65—the Epitope of Human Prostate Specific Antigen Fusion Protein(HSP65-PSAe)

The target cells were prepared by the following procedure:

B16 cells were transfected with recombinant VR1055 plasmids carrying three copies of the epitope of PSA encoding genes, cultured in 10% FBS IMDM containing G418 (500 μg/ml), and B16 cells expressing the epitope of PSA (MEPSA) were identified by RT-PCR and Western blot analysis.

B16 cells transfected with MEPSA gene were cultured in 10% FBS IMDM containing 10% Con A conditioned medium in the atmosphere of 5% CO₂ at 37 for 24 hours. ×10⁶ B16 cells transfected with MEPSA gene were labeled with 100 μCi ⁵¹Cr in the atmosphere of 5% CO₂ at 37 for 24 hours. The cells were washed with serum free IMDM four times, 2 ml each time. The washed cells were suspended in 10 ml 10% FBS IMDM, and 100 μl of the cell suspension was added into one well of round bottom 96-well plate.

The effector cells were prepared by the following procedure: Six 8-week-old male C57BL/6 mice were injected with 10 μg of HSP65-PSAe in 200 μl PBS on day 0, 14 and 28. Control groups were injected with PBS. The spleen cells were removed 5 days after the last immunization and cultured in 10% FBS IMDM containing 10% Con-A conditioned medium and HSP65-PSAe (10 μg/ml) in the atmosphere of 5% CO₂ at 37 for 7 days and then subjected to evaluate CTL responses. The ConA Conditioned medium was prepared by the following procedure The spleen cells were isolated from a C57BL/6 mouse and cultured with 6 ml 10% FBS IMDM containing ConA (final concentration is 5mg/ml) in the atmosphere of 5% CO₂ at 37 for 24 hours. The supernatant was collected as Con A conditioned medium. The splenocytes within a group were pooled. Serial dilutions of resultant spleen cells were cultured with 1×10⁴ transfected B 16 cells labeled with ⁵¹Cr in 200 μl round bottom well of 96 well plate. After 10 hours incubation in the atmosphere of 5% CO₂ at 37 , the plate was centrifuged at 3000 rpm for 5 minutes and then 100 μl supernatant was collected for gamma radiation counting. The percent specific release was calculated by the formula of ((specific release-spontaneous release)/(total release-spontaneous release))×100%. The spontaneous release was less than 15%.

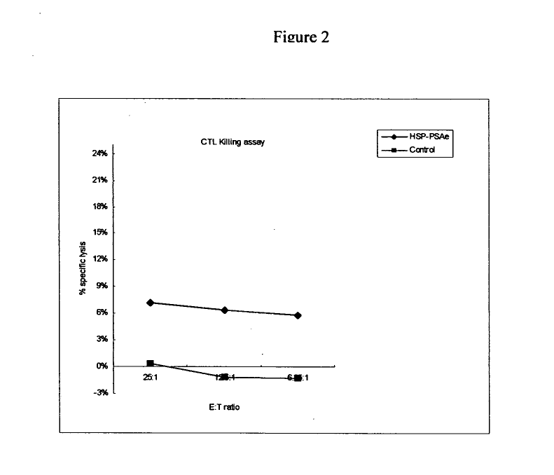

The results were showed in FIG. 2.

Conclusion: Bacillus Calmette Guérin heat shock protein 65—the epitope of human prostate specific antigen fusion protein can induce PSA specific cytotoxic T lymphocytes in mice, and the CTL can kill tumor cells expressing PSA specific epitopes.

### Example 5

The growth of B16 cells transfected with MEPSA gene was inhibited in mice inoculated with Bacillus Calmette Guérin heat shock protein 65—the epitope of human prostate specific antigen fusion protein.

8-week-old male C57BL/6 mice were injected subcutaneously with 5 μg of HSP65-PSAe in 200 μl PBS on day 0, 14 and 28. Control groups were injected with PBS. 15 mice were included in each group. In 5 days after the last immunization, the mice were injected with 1 10⁵ B16 cells transfected with MEPSA gene subcutaneously in the back near the hind leg. Two weeks later, the tumor nodule was palpated and measured two dimensionally daily.

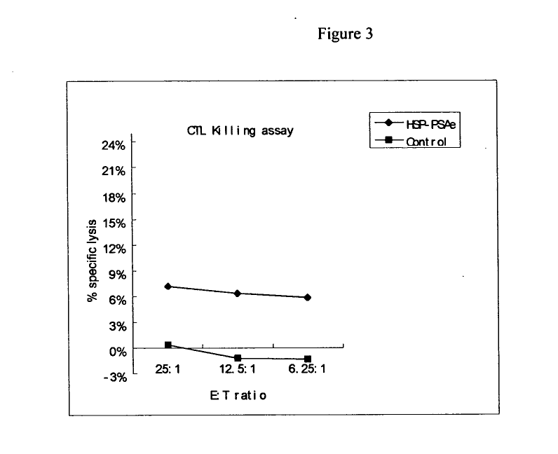

The results were showed in FIG. 3.

It was concluded that injection of Bacillus Calmette Guérin heat shock protein 65—the epitope of human prostate specific antigen fusion protein induced the growth inhibition of B16 cells transfected with MEPSA gene in mice.

### Example 6

### Induction of Human PSA Specific CTLs in vitro by Immunization of HSP65-PSAe Loaded Autologous Dendritic Cells

Human peripheral blood mononuclear cells (PBMC) were isolated from HLA-A2+ donor blood by Ficoll-hypaque (BD, Pharmingen) and Percoll (BD, Pharmingen) centrifugation successively. The PBMC were diluted with 10% IMDM to 2×10⁶/ml. 1 ml of the cell suspension was added into one well of 12-well plate with the cell final concentration of 1×10⁶. After 2-hours adherence in the atmosphere of 5% CO₂ at 37 , the non-adherent cells were removed from the wells. The cells were washed twice by using serum free IMDM with gently shaking and cultured in 2 ml of 10% FBS IMDM supplemented with 10 ng (200 U/ml) GM-CSF and 200 U/ml IL4 in the atmosphere of 5% CO₂ at 37 for 5 days. On day 5, after HSP65-PSAe was added (100 μg/ml), the cells were cultured for another 2 days. The induced dendritic cells were harvested on the seventh day and used immediately or frozen in liquid nitrogen.

Isolation of CD8⁺ T cells from PBMC

PBMCs (20×10⁶ cells/ml) isolated from human (HLA-A2⁺) blood buffy coat were incubated in 25 ml PBS/EDTA/human serum buffer with a monoclonal antibody cocktail against CD56, CD19 and CD4 on ice for 30 minutes with shaking. After being washed by centrifugation twice, the cells were incubated with Anti-mouse IgG magnetic beads at the ratio of cells:beads=1:4 on ice for 20 minutes, and then antibody coated cells were removed by using a magnet. 90% resultant cells were identified as CD8⁺ cells.

Isolated CD8⁺ cells were in vitro immunized by autologous DCs loaded with HSP65-PSAe on day 0, 7 and 14. On day 7 after the last immunization, the effector cells were seeded in 200 μl round bottom well of 96 well plate and co-cultured with 1×10^{4 51}Cr labeled T2 cells loaded with PSA derived peptides (FLTPKKLQCV) at increasing effector target ratios for 4 hours. The plate was centrifuged at 3000 rpm for 5 minutes and then 100 μl supernatant was collected for gamma radiation counting. The percent specific release was calculated by the formula of ((specific release-spontaneous release)/(total release-spontaneous release))×100. The spontaneous release was less than 15%.

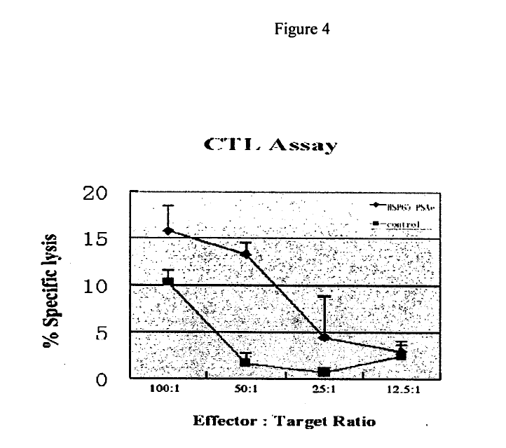

The results were shown in FIG. 4.

1. A recombinant fusion protein comprising Bacillus Calmette Guérin heat shock protein 65 and one to five copies of an epitope of human prostate specific antigen.

2. A nucleic acid molecule encoding the recombinant fusion protein of claim 1.

3. A composition comprising the recombinant fusion protein of claim 1 as the active ingredient and a pharmaceutically acceptable excipient.

4. A method of making a medicament, comprising preparing a pharmaceutical preparation comprising the recombinant fusion protein of claim 1 and a biologically acceptable excipient.

5. The recombinant fusion protein according to claim 1, wherein the Bacillus Calmette Guérin heat shock protein 65 is located at an N-terminus of the fusion protein, and the epitope of human prostate specific antigen is located at a C-terminus of the fusion protein.

6. A nucleic acid molecule encoding the recombinant fusion protein of claim 5.

7. A composition comprising the recombinant fusion protein of claim 5 as the active ingredient and a pharmaceutically acceptable excipient.

8. A method of making a medicament, comprising preparing a pharmaceutical preparation comprising the recombinant fusion protein of claim 5 and a biologically acceptable excipient.

9. The recombinant fusion protein of claim 1 wherein the epitope of human prostate specific antigen comprises an amino acid sequence having at least 85% identity to a member of the group consisting of SEQ ID NO: 2 and SEQ ID NO: 4.

10. A nucleic acid molecule encoding the recombinant fusion protein of claim 9.

11. A composition comprising the recombinant fusion protein of claim 9 as the active ingredient and a pharmaceutically acceptable excipient.

12. A method of making a medicament, comprising preparing a pharmaceutical preparation comprising the recombinant fusion protein of claim 9 and a biologically acceptable excipient.

13. The recombinant fusion protein of claim 9, wherein the epitope of human prostate specific antigen comprises an amino acid sequence in the group consisting of SEQ ID NO: 2 and SEQ ID NO: 4.

14. A nucleic acid molecule encoding the recombinant fusion protein of claim 13.

15. The recombinant fusion protein according to claim 1, comprising an amino acid sequence having at least 85% identity to a member of the group consisting of SEQ ID NO: 6, and SEQ ID NO: 8.

16. A nucleic acid molecule encoding the recombinant fusion protein of claim 15.

17. A composition comprising the recombinant fusion protein of claim 15 as the active ingredient and a pharmaceutically acceptable excipient.

18. A method of making a medicament, comprising preparing a pharmaceutical preparation comprising the recombinant fusion protein of claim 15 and a biologically acceptable excipient.

19. The recombinant fusion protein according to claim 15 comprising an amino acid sequence in the group consisting of SEQ ID NO: 6 and SEQ ID NO: 8.

20. A nucleic acid molecule encoding the recombinant fusion protein of claim 19.

21. A recombinant fusion protein comprising Bacillus Calmette Guérin heat shock protein 65, one to five copies of an epitope of human prostate specific antigen, with the protein being encoded by a nucleic acid molecule comprising a nucleic acid sequence having at least 85% identity to SEQ ID NO: 5 or SEQ ID NO: 7.

22. The recombinant fusion protein of claim 21, wherein the Bacillus Calmette Guérin heat shock protein 65 is located at an N-terminus of the fusion protein, and the epitope of human prostate specific antigen is located at a C-terminus of the fusion protein.

23. The recombinant fusion protein of claim 21, wherein the epitope of human prostate specific antigen comprises an amino acid sequence having at least 85% identity to a member of the group consisting of SEQ ID NO: 2 and SEQ ID NO: 4.

24. The recombinant fusion protein of claim 23, wherein the epitope of the human prostate specific antigen has a length of at least six amino acids.

25. The recombinant fusion protein of claim 23, wherein the epitope of human prostate specific antigen comprises an amino acid sequence of the group consisting of SEQ ID NO: 2 and SEQ ID NO: 4.

26. The recombinant fusion protein of claim 21 comprising an amino acid sequence having at least 85% identity to a member of the group consisting of SEQ ID NO: 6 and SEQ ID NO: 8.

27. The recombinant fusion protein of claim 26 wherein the amino acid sequence is a member of the group consisting of SEQ ID NO: 6 and SEQ ID NO: 8.

28. The recombinant fusion protein of claim 21, wherein the nucleic acid molecule has at least 95% identity to SEQ ID NO: 5 or SEQ ID NO: 7.

29. A plasmid, the plasmid comprising a recombinant fusion protein that comprises Bacillus Calmette Guérin heat shock protein 65 and one to five copies of an epitope of human prostate specific antigen.

30. A host cell transformed with the plasmid of claim 29.

31. The plasmid of claim 29, wherein the Bacillus Calmette Guérin heat shock protein 65 is located at an N-terminus of the recombinant fusion protein, and the epitope of human prostate specific antigen is located at a C-terminus of the fusion protein.

32. The plasmid of claim 29, wherein wherein the epitope of human prostate specific antigen comprises an amino acid sequence having at least 85% identity to a member of the group consisting of SEQ ID NO: 2 and SEQ ID NO: 4.

33. The plasmid of claim 32, wherein the epitope of human prostate specific antigen comprises an amino acid sequence that is a member of the group consisting of SEQ ID NO: 2 and SEQ ID NO: 4.

34. The plasmid of claim 29 comprising an amino acid sequence having at least 85% identity to a member of the group consisting of SEQ ID NO: 6 and SEQ ID NO: 8.

35. The plasmid of claim 34 wherein the amino acid sequence is a member of the group consisting of SEQ ID NO: 6 and SEQ ID NO: 8.
